(19) United States
(12) Patent Application Publication
(10) Pub. No.: US 2022/0324721 A1
(43) Pub. Date: Oct. 13, 2022

(54) SENSING MATERIAL FOR DETECTING HYDROGEN SULFIDE, HYDROGEN SULFIDE-SENSITIVE LAYER, AND METAL OXIDE SEMICONDUCTOR-TYPE GAS SENSOR

(71) Applicant: TDK CORPORATION, Tokyo (JP)

(72) Inventors: Tomokazu ISHIKURA, Tokyo (JP); Raitaro MASAOKA, Tokyo (JP)

(73) Assignee: TDK CORPORATION, Tokyo (JP)

(21) Appl. No.: 17/678,086

(22) Filed: Feb. 23, 2022

(30) Foreign Application Priority Data
Mar. 31, 2021 (JP) 2021-062270

(51) Int. Cl. C01G 49/00; G01N 27/12; G01N 33/00

(52) U.S. Cl. CPC C01G 49/0018; G01N 27/125; G01N 33/0044; C01P 2004/61

(57) ABSTRACT

A sensing material for detecting hydrogen sulfide capable of detecting hydrogen sulfide even having a low concentration, a hydrogen sulfide-sensitive layer containing the sensing material for detecting hydrogen sulfide, and a metal oxide semiconductor-type gas sensor having the hydrogen sulfide-sensitive layer are provided. The sensing material for detecting hydrogen sulfide includes CuFe₂O₄-type complex oxide (W). The CuFe₂O₄-type complex oxide (W) contains, as a main component (W1), 35.0 to 49.5 mol % of iron oxide in terms of Fe₂O₃ and 50.5 to 65 mol % of copper oxide in terms of CuO, and an average particle diameter of particles is 3 μm or less.

### BACKGROUND OF THE INVENTION

### Field of the Invention

The disclosure provides a sensing material for detecting hydrogen sulfide, a hydrogen sulfide-sensitive layer, and a metal oxide semiconductor-type gas sensor.

Priority is claimed on Japanese Patent Application No. 2021-062270, filed Mar. 31, 2021, the content of which is incorporated herein by reference.

### Description of Related Art

Hydrogen sulfide is a substance causing offensive odors. As a sensor for detecting hydrogen sulfide, for example, sensors using a potentiostatic electrolysis sensor method, a non-dispersive infrared analysis method, gas chromatography, ion chromatography, and the like are used. However, when a potentiostatic electrolysis sensor is used, since a liquid such as an electrolyte is used, the apparatus is complicated, and maintenance management such as replenishment of the liquid must be performed frequently. Further, when the non-dispersive infrared analysis method is used, it has a disadvantage in that the required apparatus is expensive and large. Furthermore, when gas chromatography or ion chromatography is used, the apparatus becomes large and expensive, and the concentration of hydrogen sulfide cannot be continuously measured.

Patent Document 1 provides a semiconductor-type hydrogen sulfide gas sensor which is made of a tin oxide semiconductor to which oxides of lanthanum and lead are added as additives. However, sensitivity of the sensor is insufficient when hydrogen sulfide is at a low concentration of 1 ppm or less.

On the other hand, Patent Document 2 discloses a porous film gas sensor made of a composite containing CuFe₂O₄, In₂O₃, and a noble metal element as a sensing material of the gas sensor for detecting a combustible gas, particularly carbon monoxide, in exhaust gas of various combustion equipment. It was disclosed that the sensor containing the sensing material having 10 to 50 wt % of CuFe₂O₄ with respect to In₂O₃ has a high sensitivity.

Patent Document 3 discloses a sensing material of a gas sensor containing a main component of MgFe₂O₄ and another component of 0.1 to 45 mol % of Cr₂O₃, which is used as a sensing material of a gas sensor for detecting the presence of a reducing gas such as LP gas (propane) by detecting a change of electric resistance.

### PATENT DOCUMENTS

[Patent Document 1] Japanese Patent No. 2686384
[Patent Document 2] Japanese Patent Application Laid-Open No. Hei 10-115597
[Patent Document 3] Japanese Patent Application Laid-Open No. 58-048854

### SUMMARY OF THE INVENTION

An object of the present disclosure is to provide a metal oxide semiconductor-type gas sensor capable of detecting hydrogen sulfide contained in a gas to be measured at a concentration of 0.5 ppm or less.

According to the present disclosure, the following are provided.

[1] A sensing material for detecting hydrogen sulfide, which comprises CuFe₂O₄-type complex oxide (W),

wherein the CuFe₂O₄-type complex oxide (W) comprises, as a main component (W1), 35.0 to 49.5 mol % of iron oxide in terms of Fe₂O₃ and 50.5 to 65 mol % of copper oxide in terms of CuO, and

an average particle diameter of particles of the sensing material for detecting hydrogen sulfide is 3 μm or less.

[2] The sensing material for detecting hydrogen sulfide according to [1], wherein the CuFe₂O₄-type complex oxide (W) further comprises, as a sub-component (W2), at least one selected from the group consisting of titanium oxide, tin oxide, and tricobalt tetroxide, and

a total content of the sub-component (W2) is 20 mass % or less in terms of TiO₂, SnO₂, and CoO.

[3] The sensing material for detecting hydrogen sulfide according to [1], wherein the sensing material for detecting hydrogen sulfide further comprises one or more supported metals (M) selected from palladium, gold, silver, and platinum,

the CuFe₂O₄-type complex oxide (W) supports the supported metal (M), and

a total content of the supported metal (M) is 0.1 mass % or more and 25 mass % or less with respect to 100 mass % of the sensing material for detecting hydrogen sulfide.

[4] The sensing material for detecting hydrogen sulfide according to [1], wherein, when an average atomic ratio of Fe per particle in the sensing material for detecting hydrogen sulfide is set to X1 and an average atomic ratio of Cu is set to X2, and X1/X2=α, the CV value of α is 30.0% or less.

[5] A hydrogen sulfide-sensitive layer which is made of the sensing material for detecting hydrogen sulfide described in [1].

[6] A metal oxide semiconductor-type gas sensor, comprising:

a substrate;

an electrode formed on the substrate;

the hydrogen sulfide-sensitive layer as described in [5], which is formed on a surface of the substrate on which the electrode is formed.

The present disclosure provides a sensing material for detecting hydrogen sulfide which is excellent in gas adsorptivity to a target gas and which can be detected with higher sensitivity, as well as a metal oxide semiconductor-type gas sensor.

### BRIEF DESCRIPTION OF THE DRAWINGS

FIG. 1 is a schematic view showing a metal oxide semiconductor-type gas sensor according to an embodiment of the present invention.

FIG. 2 is a schematic view showing a cross-sectional view of a metal oxide semiconductor-type gas sensor according to an embodiment of the present invention (line A-A′ in FIG. 1).

FIG. 3 is a graph showing sensitivity [Ra/Rg] of a hydrogen sulfide sensor and concentration [ppm] of hydrogen sulfide in Example 8 and Comparative Examples 2 and 3.

### DETAILED DESCRIPTION OF THE INVENTION

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

<Sensing Material for Detecting Hydrogen Sulfide>

### First Embodiment

The sensing material for detecting hydrogen sulfide of the present embodiment includes CuFe₂O₄-type complex oxide (W). The sensing material for detecting hydrogen sulfide of the present embodiment is preferably CuFe₂O₄-type complex oxide (W). The CuFe₂O₄-type complex oxide (W) contains, as a main component (W1), 35.0 to 49.5 mol % of iron oxide in terms of Fe₂O₃ and 50.5 to 65 mol % of copper oxide in terms of CuO.

In the CuFe₂O₄-type complex oxide (W), iron oxide as the main component (W1) is preferably 37.0 to 48.0 mol % in terms of Fe₂O₃, and more preferably 42.0 to 47.0 mol %. Copper oxide is preferably 52.0 to 63 mol % in terms of CuO, and more preferably 53.0 to 58 mol %. When copper oxide is contained in an amount of 50.5 mol % or more in terms of CuO, since CuO, which is a p-type semiconductor oxide, exists, a p-n heterojunction is formed at the interface between CuO and CuFe₂O₄ which is an n-type semiconductor oxide and which has a stoichiometric composition, and sensitivity is improved. On the other hand, when copper oxide is contained in an amount of more than 65 mol % in terms of CuO, abnormal grain growth is observed in a part at the time of preliminary firing (heat treatment), and subsequent grinding becomes difficult. As a result, sensitivity decreases. The main component (W1) of the CuFe₂O₄-type complex oxide (W) is the sum of a component derived from iron oxide and a component derived from copper oxide. The conversion molar ratio is a molar ratio with respect to 100 mol % of the main component (W1).

“Average Particle Diameter”

The average particle diameter of particles of the sensing material for detecting hydrogen sulfide in the present embodiment is 3.0 μm or less. It is preferably 2.0 μm or less, and more preferably 1.7 μm or less. This is because when the average particle diameter of particles of the sensing material for detecting hydrogen sulfide of the present embodiment is 3.0 μm or less, the surface area where the gas is adsorbed is increased and the sensitivity is improved. Further, from the viewpoint of the possibility of synthesizing particles of the sensing material for detecting hydrogen sulfide of the present embodiment or the workability in fabricating a hydrogen sulfide-sensitive layer to be described later, the average particle diameter of particles of the sensing material for detecting hydrogen sulfide of the present embodiment is preferably 10 nm or more, and more preferably 50 nm or more.

The method of evaluating the average particle diameter will be described in detail in the examples.

“CV Value of Composition”

When an average atomic ratio of Fe per particle in the sensing material for detecting hydrogen sulfide of the present embodiment is set to X1 and an average atomic ratio of Cu is set to X2, and X1/X2=α, the CV value of α (coefficient of variation=standard deviation/average) is preferably 30.0% or less, and more preferably 25.0% or less.

X1/X2 is a value obtained by dividing the content of Fe by the content of Cu in one particle. The average value of α is the average value of α for each particle. The average value of α approximately corresponds to the value obtained by dividing the Fe content by the Cu content of the whole powder based on the number of atoms. The CV value of α is a parameter representing the dispersion of α in each particle, that is, the dispersion of the composition between particles. The smaller the CV value of α is, the smaller the dispersion of α is, and the smaller the dispersion of the composition between particles is.

The CuFe₂O₄-type complex oxide (W) powder according to the present embodiment has good detection sensitivity when the CV value of α is 30.0% or less.

Methods for measuring X1 and X2 are not particularly limited. For example, STEM-EDX or the like can be used to perform point analysis on 5 or more points, preferably 10 or more points in one particle, to measure the atomic ratio of Fe and the atomic ratio of Cu at each point, and to calculate X1 and X2 by averaging the data. There is no particular limit to the observation magnification of STEM. For example, it may be about 20000 to 40000 times. It is difficult to properly measure X1 and X2 when the observation magnification of STEM is too high or too low. The spot diameter (hereinafter referred to simply as beam diameter) of the electron beam on the surface of the measurement sample for point analysis is appropriately set according to the observation magnification of the STEM. For example, it may be about 0.2 to 1.0 nm. In order to set the beam diameter within the above range, for example, a STEM having a field emission-type electron gun may be used.

There are no particular restrictions on the method of calculating the average value of α and the CV value of α. For example, α can be calculated for 10 or more particles, preferably 30 or more particles contained in the powder, and the average value of a can be calculated by averaging them. Furthermore, the standard deviation of α is calculated, and the CV value of α can be calculated by dividing the standard deviation of α by the average value of α.

The method of evaluating the CV value of α will be described in detail in the examples.

As used herein, a “CuFe₂O₄-type complex oxide” refers to a complex oxide having a composition similar to that of a conventional CuFe₂O₄ complex oxide (W₀) with a molar composition ratio of Cu/Fe/O=1:2:4 of elemental Cu, Fe, and oxygen and having a crystal structure similar to that of the CuFe₂O₄ complex oxide (W₀).

For example, a CuFe₂O₄-type complex oxide (W) according to an embodiment of the present invention contains a certain amount of excess Cu compared to a conventional CuFe₂O₄-type complex oxide (W₀). In addition, the CuFe₂O₄-type complex oxide (W) of another embodiment includes metal components other than the main components of Cu and Fe.

When the CuFe₂O₄-type complex oxide (W) of the present invention and the conventional CuFe₂O₄ complex oxide (W₀) contain different composition ratios or different metal species, for example, when in comparison with the conventional CuFe₂O₄ complex oxide (W₀), the CuFe₂O₄-type complex oxide (W) of the present invention contains Cu in excess and contains different metal species Ti or the like, and these Cu or metal Ti or the like may replace the existing metal species in the crystal structure of the conventional CuFe₂O₄ complex oxide (W₀), or may exist in the crystal structure as another oxide crystalline phase existing separately from the conventional crystal, and may exist as another oxide crystal or an amorphous material between the primary particles of the conventional CuFe₂O₄ complex oxide (W₀). Alternatively, a combination thereof may be used.

“Method of Manufacturing Sensing Material for Detecting Hydrogen Sulfide of the Embodiment”

Next, an example of a method for producing the sensing material for detecting hydrogen sulfide according to the present embodiment will be described. First, a starting raw material (raw material of main component) is prepared. The raw material of the main component is not particularly limited, but the following is preferably used.

Iron oxide (α-Fe₂O₃), copper oxide (CuO), or a complex oxide of them can be used as a raw material of the main component.

Further, various compounds or the like which can become the above-mentioned oxides or complex oxide by firing them can be used. Examples of the compounds which can become the above-mentioned oxides by firing them include a single metal, a carbonate, an oxalate, a nitrate, a hydroxide, a halide, an organometallic compound, and the like.

First, the prepared starting material is weighed and mixed so as to have a predetermined composition ratio to obtain a raw material mixture. Examples of the mixing method include wet mixing using a ball mill and dry mixing using a dry mixer. It is preferable to use a starting material having an average particle diameter of 0.1 to 3 μm.

Next, the raw material mixture is subjected to heat treatment to obtain a heat treatment material. The heat treatment is carried out in order to cause loss of ultrafine powder and grain growth to a proper particle diameter by pyrolysis of the raw material, homogenization of the components, formation of the sensing material for detecting hydrogen sulfide and sintering it, and to convert the raw material mixture to a form suitable for the post-process. Such heat treatment is preferably carried out at a temperature of 600 to 1000° C., usually for about 1 to 3 hours. The heat treatment may be carried out in the atmosphere (air), or under a condition having an oxygen partial pressure lower than that in the atmosphere or in a pure oxygen.

Next, the heat treatment material is pulverized to obtain a pulverized material. The pulverization is carried out in order to break the agglomeration of the heat treatment material into a powder having proper sinterability. When the heat-treated material forms a large mass, wet grinding is performed by using a ball mill or an attritor after rough grinding is performed. The wet grinding is carried out until the average particle diameter of the heat-treated material is preferably about 0.05 to 3 μm.

A sensing material for detecting hydrogen sulfide having a predetermined average particle diameter can be obtained by appropriately adjusting a heat treatment temperature, a grinding time, etc.

### Second Embodiment

The sensing material for detecting hydrogen sulfide of the present embodiment includes CuFe₂O₄-type complex oxide (W). The sensing material for detecting hydrogen sulfide of the present embodiment is preferably CuFe₂O₄-type complex oxide (W). The CuFe₂O₄-type complex oxide (W) of the present embodiment can contain at least one selected from the group consisting of titanium, tin, and cobalt elements as a secondary component (W2) in addition to the main component (W1) of the first embodiment. The total content of the sub-component (W2) is preferably 20 mass % or less in terms of TiO₂, SnO₂, and CoO, more preferably 15 mass % or less, and still more preferably 100 mass % or less with respect to 10 mass % of the CuFe₂O₄-type complex oxide (W).

“Average Particle Diameter”

The average particle diameter of particles of the sensing material for detecting hydrogen sulfide of the present embodiment is the same as that of the first embodiment.

“CV Value of Composition”

The CV value of the composition of the sensing material for detecting hydrogen sulfide of the present embodiment is the same as that of the first embodiment.

“Method of Manufacturing Sensing Material for Detecting Hydrogen Sulfide of the Present Embodiment”

An example of a method for manufacturing a sensing material for detecting hydrogen sulfide according to the present embodiment will be described. First, a starting raw material (the raw materials of the main component and the secondary component) is prepared. The raw material of the main component and the raw material of the secondary component are not particularly limited, but the following materials are preferably used.

Iron oxide (α-Fe₂O₃), copper oxide (CuO), titanium oxide (TiO₂), tin oxide (SnO₂), tricobalt tetroxide (Co₃O₄), or a complex oxide of them can be used as a raw material for the main and secondary components.

Further, various compounds or the like which can become the above-mentioned oxides or complex oxide by firing them can be used. Examples of the above-mentioned compounds which can become the above-mentioned oxides by firing them include a single metal, a carbonate, an oxalate, a nitrate, a hydroxide, a halide, an organometallic compound, and the like.

First, the prepared starting material is weighed and mixed so as to have a predetermined composition ratio to obtain a raw material mixture. Examples of the mixing method include wet mixing using a ball mill and dry mixing using a dry mixer. It is preferable to use a starting material having an average particle diameter of 0.1 to 3 μm.

Next, the raw material mixture is subjected to heat-treatment to obtain a heat-treated material. The heat treatment is carried out in order to cause loss of ultrafine powder and grain growth to a proper particle diameter by pyrolysis of the raw material, homogenization of the components, formation of the sensing material for detecting hydrogen sulfide and sintering it, and to convert the raw material mixture to a form suitable for the post-process. Such heat treatment is preferably carried out at a temperature of 600 to 1000° C., usually for about 1 to 3 hours. The heat treatment may be carried out in the atmosphere (air), or under a condition having an oxygen partial pressure lower than that in the atmosphere or in a pure oxygen.

Next, the heat treatment material is pulverized to obtain a pulverized material. The pulverization is carried out in order to break the agglomeration of the heat treatment material into a powder having proper sinterability. When the heat-treated material forms a large mass, wet grinding is performed by using a ball mill or an attritor after rough grinding is performed. The wet grinding is carried out until the average particle diameter of the heat-treated material is preferably about 0.05 to 3 μm.

### Third Embodiment

As in the first or second embodiment, the sensing material for detecting hydrogen sulfide of the present embodiment includes the CuFe₂O₄-type complex oxide (W). In addition to the CuFe₂O₄-type complex oxide (W), the sensing material for detecting hydrogen sulfide of the present embodiment further includes one or more supported metals (M) selected from the group consisted of palladium, gold, silver, and platinum. The CuFe₂O₄-type complex oxide (W) supports the supported metal (M). The sensing material for detecting hydrogen sulfide of the present embodiment is preferably a CuFe₂O₄-type complex oxide (W) supporting the supported metal (M). With respect to 100 mass % of the sensing material for detecting hydrogen sulfide, the total content of the supported metal (M) is 0.1 mass % or more and 25 mass % or less. The total content of the supported metal (M) is preferably 1 mass % or more and 20 mass % or less, and the total content of the supported metal (M) is more preferably 5 mass % or more and 15 mass % or less.

“Average Particle Diameter”

The average particle diameter of particles of the sensing material for detecting hydrogen sulfide of the present embodiment is the same as that of the first embodiment.

“CV Value of Composition”

The CV value of the composition of the sensing material for detecting hydrogen sulfide of the present embodiment is the same as that of the first embodiment.

“Method of Manufacturing Sensing Material For Detecting Hydrogen Sulfide of the Present Embodiment”

First, as in the first embodiment, a powder of a CuFe₂O₄-type complex oxide containing only a main component or, as in the second embodiment, a powder of a CuFe₂O₄-type complex oxide containing a main component and sub-components is produced as a precursor of the sensing material for detecting hydrogen sulfide of the present embodiment.

Then, the powder of the CuFe₂O₄-type complex oxide as the precursor of the sensing material for detecting hydrogen sulfide of the present embodiment is dispersed in ethanol as a dispersion medium, and a noble metal colloid containing each supported metal (M) is added so that the total content of one or more supported metals (M) selected from the group consisted of palladium, gold, silver, and platinum is 0.1 mass % or more and 25 mass % or less while the dispersion is stirred with a stirrer. Then, the dispersion medium is evaporated by heating the mixture to prepare a powder of a CuFe₂O₄-type complex oxide supporting a predetermined supported metal (M).

<Hydrogen Sulfide-Sensitive Layer>

The hydrogen sulfide-sensitive layer of the present embodiment includes any one of the sensing material for detecting hydrogen sulfides of the present disclosure, preferably any one of the sensing material for detecting hydrogen sulfides of the first to third embodiments described above. Further, for example, a binder of polyvinyl butyral resin (BM-S manufactured by Sekisui Chemical Industry Co., Ltd.) may be contained. In addition, additives such as other dispersants may be included.

[Preparation Method of Hydrogen Sulfide-Sensitive Layer]

The sensing material for detecting hydrogen sulfide (W), the dispersion medium, and, if necessary, a binder component or other additive are mixed and dispersed using a dispersing device such as a crusher (mixer mill) to prepare a paste of the sensing material for detecting hydrogen sulfide.

The particle diameter of the sensing material for detecting hydrogen sulfide (W) of the present embodiment is preferably in the range of 0.05 to 1.7 μm.

Examples of the dispersion medium include water, methanol, ethanol, diethylene glycol monobutyl ether, and the like; and mixed solvents thereof.

Examples of the binder component include polyvinyl butyral resin, polyvinyl acetal resin, polyvinyl alcohol resin, and the like.

Examples of the other additives include dispersants, wetting agents, and the like.

Examples of the dispersion method include a normal temperature crusher (mixer mill) and the like.

Examples of the dispersion condition include a stirring time of 20 minutes to 5 hours and the like.

The paste is applied to the gas adsorption part of various hydrogen sulfide gas sensors, dried, and debinder-treated to form a hydrogen sulfide-sensitive layer. The thickness of the hydrogen sulfide-sensitive layer is not particularly limited, but may be appropriately selected depending on the type of the hydrogen sulfide gas sensor, the amount of analysis, and the like. In the case of the metal oxide semiconductor-type gas sensor described later, 2 μm to 500 μm is preferable, and 5 μm to 20 μm is more preferable.

The dispersion method and the dispersion condition, the coating method and the coating condition, and the drying method and the drying condition are not particularly limited, and, for example, the following methods and conditions can be used.

Examples of the coating method include a coating method using a dispenser such as a pneumatic dispenser, a tubing dispenser, a volumetric dispenser, and the like.

Examples of the drying method and condition include a method of drying by heating at 50 to 150° C. in air, and the like.

Examples of the debinding condition include a method of firing at 200 to 600° C. for 1 to 20 hours in air using a firing furnace, and the like.

<Metal Oxide Semiconductor-Type Gas Sensor>

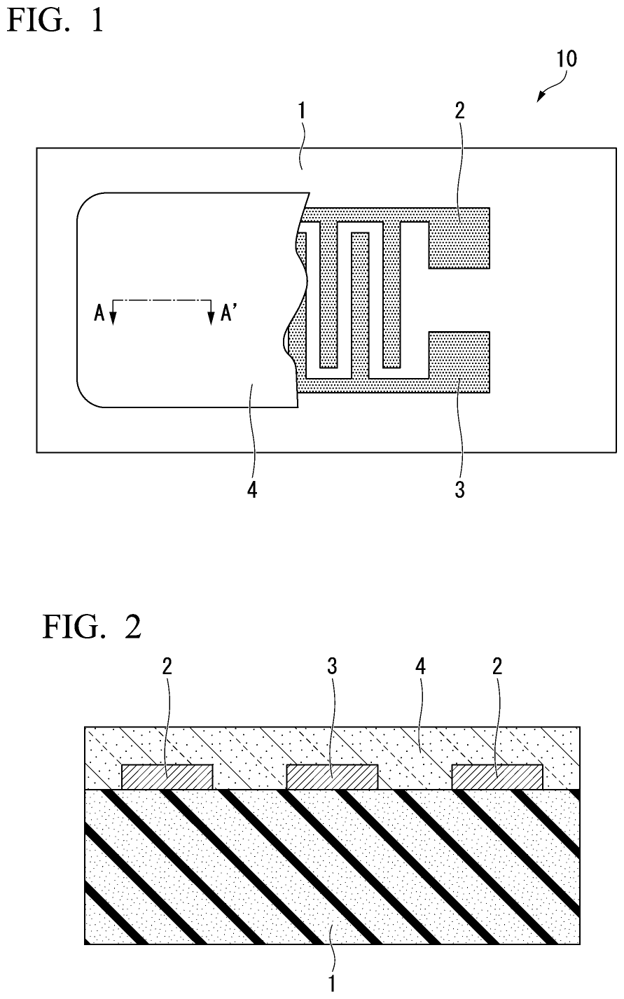

In the metal oxide semiconductor-type gas sensor of the present embodiment, as shown in FIGS. 1 and 2, a hydrogen sulfide-sensitive layer made of a sensing material for detecting hydrogen sulfide is arranged between comb-type electrodes provided on a substrate, and the amount of hydrogen sulfide adsorbed is detected by detecting a change of electric resistance between the electrodes by applying a voltage between the electrodes. The sensing material for detecting hydrogen sulfide between the electrodes provided on the substrate constitutes the hydrogen sulfide-sensitive layer of the present disclosure.

Since the metal oxide semiconductor-type gas sensor of the present embodiment uses the sensing material for detecting hydrogen sulfide of the present disclosure, the sensitivity of the electric resistance change after adsorption of hydrogen sulfide is high. For this reason, by using the metal oxide semiconductor-type gas sensor of the present disclosure, it is possible to detect hydrogen sulfide in a minute concentration and to detect gas with high sensitivity via a simpler method.

In the metal oxide semiconductor-type gas sensor of the present disclosure, the adsorption of hydrogen sulfide is detected by detecting a change of the resistance value of a hydrogen sulfide-sensitive layer containing a sensing material for detecting hydrogen sulfide. The general principle is explained as follows.

The metal oxide semiconductor-type gas sensor uses the resistance value of the sensor when it is exposed to air as a reference. When the sensor is exposed to air prior to the start of measurement, which is used as a reference, electron-withdrawing oxygen is adsorbed on the surface of the semiconductor (gas-sensitive layer), and when an n-type semiconductor, for example, is used as the semiconductor, a space charge layer is formed in the vicinity of the semiconductor surface. As a result, a potential barrier is formed between the semiconductor surfaces, and the movement of electrons between the semiconductors is prevented. When a reducing gas such as hydrogen sulfide flows into the semiconductor surface in this state, adsorbed oxygen is consumed, and the space charge layer becomes thin, that is, the resistance value decreases. On the other hand, when the oxidizing gas flows in, the space charge layer becomes thicker, so that the resistance value increases. When a p-type semiconductor is used as the semiconductor, the resistance value decreases with respect to the oxidizing gas, and the resistance value increases with respect to the reducing gas because a reaction opposite to the case of the n-type semiconductor occurs.

The sensing material for detecting hydrogen sulfide of the present disclosure contains the CuFe₂O₄-type complex oxide (W) containing 50.5 to 65 mol % of copper oxide in terms of CuO. The amount of copper is more than that of the conventional CuFe₂O₄ (W₀). The CuFe₂O₄-type complex oxide (W) having such a specific composition exhibits high detection sensitivity to hydrogen sulfide which is a reducing gas. The conventional CuFe₂O₄ (W₀) with stoichiometric composition is an n-type semiconductor oxide, while pure copper oxide CuO is a p-type semiconductor oxide.

For example, when the CuFe₂O₄-type complex oxide (W), which is an embodiment of the present invention, is considered in a multiphase crystal structure in which a metal copper element exists as a pure copper oxide CuO crystal (excess phase) in a conventional CuFe₂O₄ (W₀) crystal (main crystal phase) of a stoichiometric composition, the reason for the improvement in sensitivity to hydrogen sulfide can be inferred as follows. That is, a p-n heterojunction may be formed at the interface of an oxide semiconductor, such as a main crystalline phase and an excess phase, and in that case the detection mechanism is changed to achieve improved sensitivity to hydrogen sulfide.

The foregoing speculation regarding the improvement of sensitivity to hydrogen sulfide is not limited in any way to the composition and crystal structure of the sensing material for detecting the hydrogen sulfide of the present disclosure. As described above, in the CuFe₂O₄-type complex oxide (W) of the present invention, the excess copper element may be introduced into the conventional CuFe₂O₄ (W₀) crystal with a stoichiometric composition and change the semiconductor characteristics of the conventional CuFe₂O₄ (W₀) crystal itself, or amorphous copper oxide CuO may be introduced between the conventional CuFe₂O₄ (W₀) crystal particles and change the characteristics of the entire complex oxide (W). Of course, there is a possibility of realizing the above various possibilities in combination.

### EXAMPLES

Hereinafter, the present invention will be described with reference to more detailed examples, but the present invention is not limited to these examples.

### Example 1

“Production of Sensing Material for Detecting Hydrogen Sulfide (W)”

First, iron oxide (α-Fe₂O₃) powder and copper oxide (CuO) powder, as the raw materials of the main component, were prepared.

Next, the prepared raw materials of the main component were weighed to obtain the composition shown in Table 1, and then were wet-mixed in a ball mill for 16 hours to obtain a raw material mixture.

Next, the obtained raw material mixture was subjected to heat treatment at 800° C. for 3 hours in air to obtain a heat-treated material, and then was wet-pulverized by a ball mill for 16 hours to obtain a powder of the sensing material for detecting hydrogen sulfide (w-1) of the present example.

The average particle diameter of the powder was evaluated by the following evaluation method, and the results are shown in Table 1.

“Manufacturing of Hydrogen Sulfide-Sensitive Layer and Metal Oxide Semiconductor-type Gas Sensor”

The powder of the sensing material for detecting hydrogen sulfide (w-1) obtained in the present example and a vehicle composed of polyvinyl butyral resin and BDG (diethylene glycol monobutyl ether) were weighed in a mass ratio of 1:3, and were stirred and mixed for 1 hour in a normal temperature mill (mixer mill) made by SPEX Company to prepare a paste (p-1) containing the powder of the sensing material for detecting hydrogen sulfide (w-1).

As a test substrate, a commercial product (Product Name: ED-IDE3-Au, Electrode Dimension: Electrode Width 5 μm, Electrode Spacing 5 μm, Thickness 200 nm) made by Micrux Technologies Company was purchased. The paste was applied to the substrate by a pneumatic dispenser, and the substrate having the paste was fired at 450° C. in air using a firing furnace to form a hydrogen sulfide gas sensitive layer (l-1) on an electrode, thereby fabricating a metal oxide semiconductor-type gas sensor (s-1) of the present example. The thickness of the hydrogen sulfide gas sensitive layer (l-1) was 10 μm±10%.

Sensitivity of the hydrogen sulfide gas sensor of the metal oxide semiconductor-type gas sensor (s-1) of the present embodiment thus obtained was measured by the following evaluation method, and the results are shown in Table 1.

“Evaluation of Sensitivity of Hydrogen Sulfide Gas Sensor”

First, a metal oxide semiconductor-type gas sensor (s-1) manufactured in the present embodiment was installed in a sample chamber with a heater for heating, and the sample holder was heated to 300° C.

Next, the nitrogen gas and the oxygen gas were mixed so as to have a flow rate ratio of 4:1 to produce synthetic air, and then the synthetic air was flown into the sample holder at a flow rate of 500 mL/min. A resistance value of the sensor was measured by the following method.

The resistance value of the sensor was measured at 10-second intervals by using a 2-terminal method using a 2700-type multi-channel DMM manufactured by Case Ray Instruments Co., Ltd. After confirming that the sensor resistance stabilized, the measurement was carried out for 200 seconds, and the average value was taken as the resistance value (Ra) of the sensor under the synthetic air.

After measuring Ra, a constant flow rate of hydrogen sulfide gas was introduced from a nitrogen-based hydrogen sulfide standard gas cylinder into nitrogen gas, and hydrogen sulfide gas was mixed into synthetic air to form a hydrogen sulfide-containing gas.

Then, the formed hydrogen sulfide-containing gas was supplied to a metal oxide semiconductor-type gas sensor, and the change of a resistance value (Rg) of the sensor with respect to the hydrogen sulfide-containing gas of 0.5 ppm hydrogen sulfide gas concentration was examined.

Rg was the sensor resistance obtained at 15 minutes after the start of exposure to hydrogen sulfide-containing gas.

“Particle Diameter Measurement Method”

The particle diameter of the individual particles in the powder was observed using a scanning microscope (made by Hitachi, Ltd., SU 5000). Specifically, SEM photographs were subjected to image processing by software to determine the boundaries of the particles, and the areas of the particles were calculated. Then, the calculated particle area was converted into a circle equivalent diameter to calculate the particle diameter. The mean value of the obtained particle diameter was taken as the mean particle diameter.

The particle diameter was calculated for 100 crystal particles.

### Examples 2 to 9

“Production of Sensing Material for Detecting Hydrogen Sulfide (W)”

Powders of the sensing material for detecting hydrogen sulfides (w-2) to (w-9) in each example were obtained by the same method as in Example 1, except that the main component materials having the compositions shown in Table 1 were used.

The average particle diameter of the powder was evaluated in the same manner as in Example 1, and the results are shown in Table 1.

The CV value of the powder of the sensing material for detecting hydrogen sulfide (w-8) of Example 8 was evaluated by the following evaluation method, and the results are shown in Table 1.

“CV Value Evaluation of Composition”

<α In One Particle>

10 spots per particle were analyzed using STEM-EDX. The observation magnification of the STEM was 32000 times, and the beam diameter was 0.5 nm. X1 was calculated by averaging the atomic concentrations of Fe at 10 locations, and X2 was calculated by averaging the atomic concentrations of Cu at 10 locations. α in one particle was calculated from X1 and X2 of one particle.

<Average of α and CV of α>

More than 30 particles were randomly selected, and α in each particle was calculated. The mean value of α and the standard deviation of α were calculated from α in each particle. Furthermore, the CV value of α was calculated by dividing the standard deviation of α by the mean value of α.

“Manufacturing of Hydrogen Sulfide-Sensitive Layer and Metal Oxide Semiconductor-type Gas Sensor”

Metal oxide semiconductor-type gas sensors (s-2) to (s-9) of Examples 2 to 9 were fabricated by forming hydrogen sulfide gas sensitive layers (l-2) to (l-9) on electrodes in the same manner as in Example 1, except that the powders of the sensing material for detecting hydrogen sulfides (w-2) to (w-9) obtained in Examples 2 to 9 were used.

Hydrogen sulfide gas sensor sensitivity of the metal oxide semiconductor-type gas sensors (s-2) to (s-9) of Examples 2 to 9 thus obtained were measured in the same manner as in Example 1, and the results are shown in Table 1.

### Comparative Examples 1 and 2

“Production of Sensing Material for Detecting Hydrogen Sulfide (W)”

Powders of the sensing material for detecting hydrogen sulfides (cw-1) to (cw-2) of Comparative Examples 1 and 2 were obtained by the same method as in Example 1, except that the main component materials having the compositions shown in Table 1 were used.

The average particle diameters of the powder were evaluated in the same manner as in Example 1, and the results are shown in Table 1 and FIG. 1.

“Manufacturing of Hydrogen Sulfide-Sensitive Layer and Metal Oxide Semiconductor-type Gas Sensor”

Metal oxide semiconductor-type gas sensors (cs-1) to (cs-2) of Comparative Examples 1 and 2 were fabricated by forming hydrogen sulfide gas sensitive layers (cl-1) to (cl-2) on the electrodes in the same manner as in Example 1, except that the powders of the sensing material for detecting hydrogen sulfides (cw-1) to (cw-2) obtained in Comparative Examples 1 and 2 were used.

Hydrogen sulfide gas sensor sensitivity of the metal oxide semiconductor-type gas sensors (cs-1) to (cs-2) of Comparative Examples 1 and 2 thus obtained were measured in the same manner as in Example 1, and the results are shown in Table 1.

### Examples 10, 37, and 38, Comparative Example 3

“Production of Sensing Material for Detecting Hydrogen Sulfide (W)”

Powders of the sensing material for detecting hydrogen sulfides (w-10), (w-37), (w-38), and (cw-3) of Examples 10, 37, and 38 and Comparative Example 3 were obtained by the same method as in Example 8 except that the heat treatment temperature and grinding time were changed.

The average particle diameters of the powder were evaluated in the same manner as in Example 1, and the results are shown in Tables 1 and 2.

The CV values of the powders of the sensing material for detecting hydrogen sulfides (w-37) and (w-38) of Examples 37 and 38 were evaluated in the same manner as in Example 8, and the results are shown in Table 2.

“Manufacturing of Hydrogen Sulfide-Sensitive Layer and Metal Oxide Semiconductor-type Gas Sensor”

Metal oxide semiconductor-type gas sensors (s-10), (s-37), (s-38), and (cs-3) of Examples 10, 37, and 38 and Comparative Example 3 were prepared by forming hydrogen sulfide gas sensitive layers (l-10), (l-37), (l-38), and (cl-3) on electrodes in the same manner as in Example 1, except that the powders of the sensing material for detecting hydrogen sulfides (w-10), (w-37), (w-38), and (cw-3) obtained in Examples 10, 37, and 38 and Comparative Example 3 were used.

Hydrogen sulfide gas sensor sensitivity of the obtained metal oxide semiconductor-type gas sensors (s-10), (s-37), (s-38), and (cs-3) of Examples 10, 37, and 38 and Comparative Example 3 were measured in the same manner as in Example 1, and the results are shown in Tables 1 and 2.

### Example 11

(Production of Sensing Material for Detecting Hydrogen Sulfide (W))

First, iron oxide (α-Fe₂O₃) powder and copper oxide (CuO) powder were prepared as the raw materials of the main component.

Titanium oxide (TiO₂) powder, tin oxide (SnO₂) powder, and tricobalt tetroxide (Co₃O₄) powder were prepared as raw materials of the secondary component.

Next, the raw materials of the prepared main and secondary components were weighed to obtain the composition shown in Table 1, and then were wet-mixed for 16 hours in a ball mill to obtain a raw material mixture.

Next, the obtained raw material mixture was subjected to heat treatment in air at 800° C. for 3 hours to obtain a heat-treated material, and then was wet-pulverized in a ball mill for 16 hours to obtain a powder of the sensing material for detecting hydrogen sulfide (w-11) of the present example.

The average particle diameter of the powder was evaluated in the same manner as in Example 1, and the result is shown in Table 1.

“Manufacturing of Hydrogen Sulfide-Sensitive Layer and Metal Oxide Semiconductor-type Gas Sensor”

A metal oxide semiconductor-type gas sensor (s-11) of the present example was fabricated by forming a hydrogen sulfide gas sensitive layer (l-11) on an electrode in the same manner as in Example 1, except that the powder of the sensing material for detecting hydrogen sulfide (w-11) obtained in the present example was used.

Hydrogen sulfide gas sensor sensitivity of the metal oxide semiconductor-type gas sensor (s-11) of the present embodiment thus obtained was measured in the same manner as in Example 1, and the result is shown in Table 1.

### Examples 12 to 21

“Production of Sensing Material for Detecting Hydrogen Sulfide (W)”

Powders of the sensing material for detecting hydrogen sulfides (w-12) to (w-21) in Examples 12 to 21 were obtained in the same manner as in Example 11, except that the raw materials of the main component and the subsidiary component raw materials having the compositions shown in Table 1 were used.

The average particle diameters of the powders were evaluated in the same manner as in Example 1, and the results are shown in Table 1.

“Manufacturing of Hydrogen Sulfide-Sensitive Layer and Metal Oxide Semiconductor-type Gas Sensor”

Metal oxide semiconductor-type gas sensors (s-12) to (s-21) of Examples 12 to 21 were fabricated by forming hydrogen sulfide gas sensitive layers (l-12) to (l-21) on electrodes in the same manner as in Example 1, except that the powders of the sensing material for detecting hydrogen sulfides (w-12) to (w-21) obtained in Examples 12 to 21 were used.

Hydrogen sulfide gas sensor sensitivity of the metal oxide semiconductor-type gas sensors (s-12) to (s-21) of Examples 12 to 21 thus obtained were measured in the same manner as in Example 1, and the results are shown in Table 1.

### Example 22

(Production of Sensing Material for Detecting Hydrogen Sulfide (W))

First, iron oxide (α-Fe₂O₃) powder and copper oxide (CuO) powder were prepared as the raw materials of the main component.

Next, the prepared raw materials of the main component were weighed to obtain the composition shown in Table 2, and then were wet-mixed in a ball mill for 16 hours to obtain a mixture of the raw materials of the main component.

Next, the obtained mixture of the raw materials was subjected to heat-treatment in air at 800° C. for 3 hours to obtain a heat-treated material, and then was wet-pulverized in a ball mill for 16 hours to obtain powder of the precursor (CuFe₂O₄ type complex oxide) of the sensing material for detecting hydrogen sulfide of the present example.

Next, the powder of the precursor of the sensing material for detecting hydrogen sulfide of the present embodiment thus obtained was dispersed in ethanol as a dispersion medium, and a noble metal colloid containing each noble metal was added so as to obtain the ratio shown in Table 2 while the dispersion was stirred with a stirrer. Then, the dispersion medium was evaporated by heating to obtain a powder of the sensing material for detecting hydrogen sulfide (w-22) of the present example, which was a CuFe₂O₄-type complex oxide supporting a noble metal catalyst.

The average particle diameter of the powder was evaluated by the following evaluation method, and the results are shown in Table 2.

“Manufacturing of Hydrogen Sulfide-Sensitive Layer and Metal Oxide Semiconductor-type Gas Sensor”

A metal oxide semiconductor-type gas sensor (s-22) of the present example was fabricated by forming a hydrogen sulfide gas sensitive layer (l-22) on an electrode in the same manner as in Example 1, except that the powder of the sensing material for detecting hydrogen sulfide (w-22) obtained in the present example was used.

Hydrogen sulfide gas sensor sensitivity of the metal oxide semiconductor-type gas sensor (s-22) of the present embodiment thus obtained was measured in the same manner as in Example 1, and the results are shown in Table 2.

### Examples 23 to 34 and 36

“Production of Sensing Material for Detecting Hydrogen Sulfide (W)”

Powders of the sensing material for detecting hydrogen sulfides (w-23) to (w-34) and (w-36) in Examples 23 to 34 and 36 were obtained in the same manner as in Example 22, except that noble metal colloid containing each noble metal was added in the ratio shown in Table 2 using the raw material of the main component and the composition shown in Table 2.

The average particle diameters of the powder were evaluated in the same manner as in Example 1, and the results are shown in Table 2.

“Manufacturing of Hydrogen Sulfide-Sensitive Layer and Metal Oxide Semiconductor-type Gas Sensor”

Metal oxide semiconductor-type gas sensors (s-23) to (s-34) and (s-36) of Examples 23 to 34 and 36 were fabricated by forming hydrogen sulfide gas sensitive layers (l-23) to (l-34) and (l-36) on the electrodes in the same manner as in Example 1, except that the powders of the sensing material for detecting hydrogen sulfides (w-23) to (w-34) and (w-36) obtained in Examples 23 to 34 and 36 were used.

Hydrogen sulfide gas sensor sensitivity of the metal oxide semiconductor-type gas sensors (s-23) to (s-34) and (s-36) of Examples 23 to 34 and 36 thus obtained were measured in the same manner as in Example 1, and the results are shown in Table 2.

### Example 35

“Production of Sensing Material for Detecting Hydrogen Sulfide (W)”

First, iron oxide (α-Fe₂O₃) powder and copper oxide (CuO) powder were prepared as the raw materials of the main component.

Titanium oxide (TiO₂) powder, tin oxide (SnO₂) powder, and tricobalt tetroxide (Co₃O₄) powder were prepared as raw materials of the secondary component.

Next, the raw materials of the prepared main and secondary components were weighed to obtain the compositions shown in Table 2, and then were wet-mixed for 16 hours in a ball mill to obtain the mixture of raw materials.

Next, the obtained mixture of raw materials was subjected to heat-treatment in air at 800° C. for 3 hours to obtain a heat-treated material, and then was wet-pulverized in a ball mill for 16 hours to obtain powder of the precursor (CuFe₂O₄ type complex oxide) of the sensing material for detecting hydrogen sulfide of the present example.

A powder of the sensing material for detecting hydrogen sulfide (w-35) of the present example, which is a CuFe₂O₄-type complex oxide supporting a noble metal catalyst, was obtained by the same method as in Example 22, except that the powder of the precursor of the sensing material for detecting hydrogen sulfide of the present example thus obtained was used.

“Manufacturing of Hydrogen Sulfide-Sensitive Layer and Metal Oxide Semiconductor-type Gas Sensor”

A metal oxide semiconductor-type gas sensor (s-35) of the present embodiment was fabricated by forming a hydrogen sulfide gas sensitive layer (l-35) on an electrode in the same manner as in Example 1, except that the powder of the sensing material for detecting hydrogen sulfide (w-35) obtained in the present example was used.

The average particle diameter of the powder was evaluated in the same manner as in Example 1, and the result is shown in Table 2.

Hydrogen sulfide gas sensor sensitivity of the metal oxide semiconductor-type gas sensor (s-35) of the present example thus obtained was measured in the same manner as in Example 1, and the result is shown in Table 2.

TABLE 1

| | | | | | | Average | | |
|---|---|---|---|---|---|---|---|---|
| | | | | | | particle | | Sensitivity |
| | Composition | | | | | diameter | | of H₂S |
| | of sensing material for | | | | | of | | sensor |
| | detecting hydrogen sulfide | | | | | sensing | CV | (300° C.) |
| Sample | [mol %] | | [wt %] | | | material | Value | 0.5 ppm |
| number | Fe₂O₃ | CuO | TiO₂ | SnO₂ | CoO | [μm] | [%] | [Ra/Rg] |
| Comparative | 32 | 68 | — | — | — | 1.7 | | 1.87 |
| Example 1 | | | | | | | | |
| Example 1 | 35 | 65 | — | — | — | 1.2 | | 2.11 |
| Example 2 | 37.4 | 63 | — | — | — | 1.3 | | 2.14 |
| Example 3 | 40.1 | 60 | — | — | — | 1.5 | | 2.15 |
| Example 4 | 42.1 | 58 | — | — | — | 1.5 | | 2.17 |
| Example 5 | 45 | 55 | — | — | — | 1.3 | | 2.19 |
| Example 6 | 46 | 54 | — | — | — | 1.7 | | 2.2 |
| Example 7 | 47 | 53 | — | — | — | 1.5 | | 2.17 |
| Example 8 | 48 | 52 | — | — | — | 1.4 | 23.6 | 2.13 |
| Example 9 | 49.5 | 51 | — | — | — | 1.6 | | 2.01 |
| Comparative | 56 | 44 | — | — | — | 1.4 | | 1.18 |
| Example 2 | | | | | | | | |
| Example 10 | 48 | 52 | — | — | — | 3 | | 2.1 |
| Comparative | 48 | 52 | — | — | — | 3.6 | | 1.95 |
| Example 3 | | | | | | | | |
| Example 11 | 48 | 52 | 20 | — | — | 2.1 | | 2.31 |
| Example 12 | 48 | 52 | 26 | — | — | 2.6 | | 2.01 |
| Example 13 | 47.2 | 53 | — | 20 | — | 2.7 | | 2.34 |
| Example 14 | 47.2 | 53 | — | 25 | — | 2.1 | | 2.02 |
| Example 15 | 46.3 | 54 | — | — | 20 | 1.8 | | 2.44 |
| Example 16 | 46.3 | 54 | — | — | 23 | 1.6 | | 2.01 |
| Example 17 | 47.5 | 53 | 14 | 6 | — | 1.4 | | 2.32 |
| Example 18 | 47.5 | 53 | 14 | — | 6 | 1.3 | | 2.21 |
| Example 19 | 47.5 | 53 | — | 12 | 4 | 1.3 | | 2.2 |
| Example 20 | 47.5 | 53 | 14 | 4 | 2 | 1.5 | | 2.31 |
| Example 21 | 47.5 | 53 | 14 | 8 | 3 | 1.4 | | 2.03 |

TABLE 2

| | Composition of sensing material for | | | | | Supported noble | | | | Average particle | | Sensitivity of H₂S |
|---|---|---|---|---|---|---|---|---|---|---|---|---|
| | detecting hydrogen sulfide | | | | | metal | | | | diameter of | CV | Sensor (300° C.) |
| Sample | [mol %] | | [wt %] | | | [wt %] | | | | sensing material | Value | 0.5 ppm |
| number | Fe₂O₃ | CuO | TiO₂ | SnO₂ | CoO | Pd | Au | Ag | Pt | [μm] | [%] | [Ra/Rg] |
| Example 22 | 46.2 | 53.8 | — | — | — | 0.1 | — | — | — | 1.4 | | 2.46 |
| Example 23 | 47.5 | 52.5 | — | — | — | 25 | — | — | — | 1.7 | | 2.40 |
| Example 24 | 48.0 | 52.0 | — | — | — | 28 | — | — | — | 1.7 | | 2.01 |
| Example 25 | 46.5 | 53.5 | — | — | — | — | 0.1 | — | — | 1.6 | | 2.54 |
| Example 26 | 46.3 | 53.7 | — | — | — | — | 25 | — | — | 1.1 | | 2.48 |
| Example 27 | 47.5 | 52.5 | — | — | — | — | 29 | — | — | 1.2 | | 2.03 |
| Example 28 | 48.2 | 51.8 | — | — | — | — | — | 0.1 | — | 1.7 | | 2.42 |
| Example 29 | 48.0 | 52.0 | — | — | — | — | — | 25 | — | 1.6 | | 2.46 |
| Example 30 | 47.2 | 52.8 | — | — | — | — | — | 29 | — | 1.6 | | 2.01 |
| Example 31 | 47.3 | 52.7 | — | — | — | — | — | — | 0.1 | 1.7 | | 2.52 |
| Example 32 | 46.9 | 53.1 | — | — | — | — | — | — | 25 | 1.2 | | 2.48 |
| Example 33 | 47.5 | 52.5 | — | — | — | — | — | — | 29 | 1.1 | | 2.05 |
| Example 34 | 47.5 | 52.5 | — | — | — | 0.2 | 0.2 | 0.2 | 0.4 | 1.1 | | 2.88 |
| Example 35 | 47.5 | 52.5 | 2 | 4 | 4 | 0.2 | 0.2 | 0.2 | 0.4 | 1.1 | | 2.96 |
| Example 36 | 47.5 | 52.5 | — | — | — | 4 | 12 | 4 | 12 | 1.3 | | 2.10 |
| Example 37 | 48.0 | 52.0 | — | — | — | — | — | — | — | 1.4 | 29.6 | 2.08 |
| Example 37 | 48.0 | 52.0 | — | — | — | — | — | — | — | 1.4 | 29.6 | 2.08 |
| Example 38 | 48.0 | 52.0 | — | — | — | — | — | — | — | 1.6 | 33.5 | 2.01 |

### Example 39

“Hydrogen Sulfide Gas Sensor Sensitivity Dependence on Concentration of Hydrogen Sulfide Gas”

The dependency of the hydrogen sulfide gas sensor sensitivity on the concentration of hydrogen sulfide gas was investigated using the metal oxide semiconductor-type gas sensors (s-8), (cs-2), and (cs-3) obtained in Example 8 and Comparative Examples 2 and 3.

First, the metal oxide semiconductor-type gas sensor (s-8) manufactured in Example 8 was installed in a sample chamber with a heater for heating, and the sample holder was heated to 300° C.

Next, the nitrogen gas and the oxygen gas were mixed so as to have a flow rate ratio of 4:1 to produce synthetic air, and then the synthetic air was flown into the sample holder at a flow rate of 500 mL/min. The resistance value of the sensor was measured by the following method.

The resistance value of the sensor was measured at 10-second intervals by using a 2-terminal method using a 2700-type multi-channel DMM manufactured by Case Ray Instruments Co., Ltd. After confirming that the sensor resistance stabilized, the measurement was carried out for 200 seconds, and the average value was taken as the resistance value (Ra) of the sensor under the synthetic air.

After measuring Ra, a constant flow rate of hydrogen sulfide gas was introduced from a nitrogen-based hydrogen sulfide standard gas cylinder into nitrogen gas, and hydrogen sulfide gas was mixed into synthetic air to form a hydrogen sulfide-containing gas.

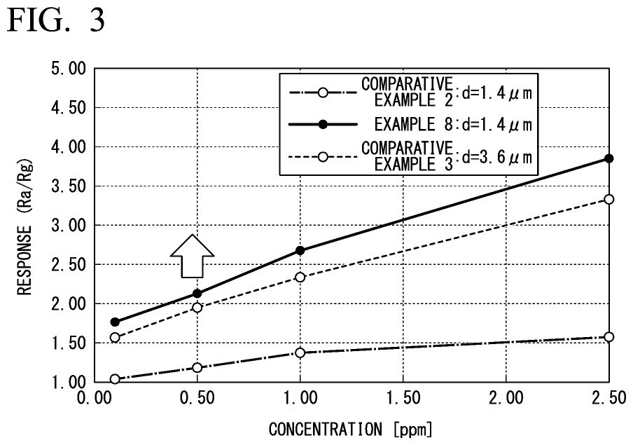

Then, the formed hydrogen sulfide-containing gas was supplied to a metal oxide semiconductor-type gas sensor, and changes of the resistance values (Rg) of the sensor with respect to hydrogen sulfide gas concentrations of 0.10 ppm, 0.50 ppm, 1.00 ppm, 2.50 ppm, and 5.00 ppm in the hydrogen sulfide-containing gas were examined. After supplying the hydrogen sulfide-containing gas of each concentration, the synthetic air was introduced, and after 15 minutes, the hydrogen sulfide-containing gas of the next concentration was supplied. Rg was the sensor resistance at 15 minutes after the start of exposure to hydrogen sulfide-containing gas. The results are shown in FIG. 3.

Similarly, using the metal oxide semiconductor-type gas sensor (cs-2) manufactured in Comparative Example 2, the dependency of the hydrogen sulfide gas sensor sensitivity on the concentration of hydrogen sulfide gas was examined in the same manner as described above. The results are shown in FIG. 3.

Similarly, using the metal oxide semiconductor-type gas sensor (cs-3) manufactured in Comparative Example 3, the dependency of the hydrogen sulfide gas sensor sensitivity on the concentration of hydrogen sulfide gas was examined in the same manner as described above. The results are shown in FIG. 3.

### SIGN DESCRIPTION

1: Substrate
2, 3: Electrode
4: Hydrogen sulfide-sensitive layer
10: Metal oxide semiconductor-type gas sensor

What is claimed is:

1. A sensing material for detecting hydrogen sulfide, which comprises CuFe₂O₄-type complex oxide (W),
wherein the CuFe₂O₄-type complex oxide (W) comprises, as a main component (W1), 35.0 to 49.5 mol % of iron oxide in terms of Fe₂O₃ and 50.5 to 65 mol % of copper oxide in terms of CuO, and
an average particle diameter of particles of the sensing material for detecting hydrogen sulfide is 3 μm or less.

2. The sensing material for detecting hydrogen sulfide according to claim 1,
wherein the CuFe₂O₄-type complex oxide (W) further comprises, as a sub-component (W2), at least one selected from the group consisting of titanium oxide, tin oxide, and tricobalt tetroxide, and
a total content of the sub-component (W2) is 20 mass % or less in terms of TiO₂, SnO₂, and CoO.

3. The sensing material for detecting hydrogen sulfide according to claim 1,
wherein the sensing material for detecting hydrogen sulfide further comprises one or more supported metals (M) selected from palladium, gold, silver, and platinum,
the CuFe₂O₄-type complex oxide (W) supports the supported metal (M), and
a total content of the supported metal (M) is 0.1 mass % or more and 25 mass % or less with respect to 100 mass % of the sensing material for detecting hydrogen sulfide.

4. The sensing material for detecting hydrogen sulfide according to claim 1,
wherein, when an average atomic ratio of Fe per particle in the sensing material for detecting hydrogen sulfide is set to X1 and an average atomic ratio of Cu is set to X2, and X1/X2=α, the CV value of α is 30.0% or less.

5. A hydrogen sulfide-sensitive layer, which is made of the sensing material for detecting hydrogen sulfide according to claim 1.

6. A metal oxide semiconductor-type gas sensor, comprising:
a substrate;
an electrode formed on the substrate; and
the hydrogen sulfide-sensitive layer according to claim 5, which is formed on a surface of the substrate on which the electrode is formed.
